(19) United States
(12) Patent Application Publication
(10) Pub. No.: US 2012/0184871 A1
(43) Pub. Date: Jul. 19, 2012

(54) EXERCISE MONITOR AND METHOD FOR MONITORING EXERCISE

(76) Inventors: Seungjin JANG, Seoul (KR); Jeongmee KOH, Seoul (KR)

(21) Appl. No.: 13/006,868

(22) Filed: Jan. 14, 2011

(51) Int. Cl. A61B 5/0488

(52) U.S. Cl. 600/546

(57) ABSTRACT

An exercise monitoring apparatus, system, and method are disclosed herein to reliably monitor an exercise using an inertial sensor and a electromyographic sensor. The sensed information may be used to determine a muscle fatigue associated with the exercised muscle and may reduce health risk factors associated with immoderate exercise. The exercise monitoring apparatus as embodied and broadly disclosed herein may include a communication unit adapted to receive muscle movement and electromyographic information from a physical information measuring apparatus. The exercise monitoring apparatus may further include a controller configured to acquire identification information related to the muscle, determine a type of exercise based upon the acquired identification information and the muscle movement related signal, and measure muscle fatigue based upon the type of exercise, the muscle movement related signal, and the electromyographic signal.

### BACKGROUND

1. Field

An exercise monitor and a method for monitoring exercise including determining muscle fatigue are disclosed herein.

2. Background

Exercise monitors and methods for monitoring exercise are known. However, they suffer from various disadvantages.

### BRIEF DESCRIPTION OF THE DRAWINGS

The embodiments will be described in detail with reference to the following drawings in which like reference numerals refer to like elements wherein:

FIG. 1 is a block diagram of an exercise monitoring system in accordance with one exemplary embodiment;

FIG. 2 is a block diagram of an exercise monitoring apparatus as shown in FIG. 1;

FIG. 3 is a block diagram of a physical information measuring apparatus as shown in FIG. 1;

FIG. 4A is a flowchart of a method for monitoring exercise in accordance with a first exemplary embodiment;

FIG. 4B is a flowchart of a method for monitoring exercise in accordance with a second exemplary embodiment;

FIG. 5 illustrates a screen that displays a level of muscle fatigue in accordance with the second exemplary embodiment;

FIG. 6 illustrates another screen that displays a level of muscle fatigue in accordance with the second exemplary embodiment;

FIG. 7A illustrates a muscle input screen in accordance with one exemplary embodiment:

FIG. 7B illustrates a process of acquiring muscle identification information in accordance with one exemplary embodiment;

FIG. 8A illustrates a muscle selection screen in accordance with one exemplary embodiment;

FIG. 8B illustrates a screen that displays a user prompt in accordance with one exemplary embodiment;

FIG. 9 illustrates a screen that displays a type of exercise in accordance with the second exemplary embodiment;

FIGS. 10A and 10B illustrate screens that display a level of muscle fatigue in accordance with the second exemplary embodiment;

FIG. 11A is a flowchart of a method for monitoring exercise in accordance with a third exemplary embodiment;

FIG. 11B is a flowchart of a method for monitoring exercise in accordance with a fourth exemplary embodiment;

FIGS. 12A and 12B illustrate outputs of the exercise monitoring apparatus according to the method for monitoring exercise in accordance with the third exemplary embodiment;

FIGS. 13A and 13B illustrate outputs of the exercise monitoring apparatus according to the method for monitoring exercise in accordance with the third exemplary embodiment;

FIGS. 14A and 14B illustrate output of the exercise monitoring apparatus according to the method for monitoring exercise in accordance with the fourth exemplary embodiment;

FIG. 15 is a flowchart of a method for monitoring exercise in accordance with a fifth exemplary embodiment;

FIG. 16 illustrates an output of the exercise monitoring apparatus according to the method for monitoring exercise in accordance with the fifth exemplary embodiment;

FIG. 17 illustrates an output of the exercise monitoring apparatus in accordance with a sixth exemplary embodiment; and

FIG. 18 is a conceptual view showing example to mount the physical information measuring apparatus according to one embodiment of the present invention.

### DETAILED DESCRIPTION

An apparatus, system, and method for monitoring physical exercise (movement, sports motion, activity, motion, or the like), as embodied and broadly described herein, may reliably monitor physical exercise and obviate or eliminate health risk factors caused by immoderate exercise by measuring muscle fatigue. A level of muscle fatigue may be based on information related to a user's physical activities as detected by an inertial sensor or an electromyographic sensor.

Muscle contraction indicates a phenomenon of muscle being contracted in response to a stimulus. For example, the muscle contraction may indicate a contraction caused due to action potentials observed at skeletal muscles of a vertebrate. Types of muscle contractions may include an isometric contraction in which a muscle is contracted while remaining the same length, an isotonic contraction in which a muscle is contracted while its length is changed, and an isokinetic contraction in which maximum muscular strength may be achieved within a Range of Motion (ROM). Also, the isotonic contraction may be categorized into an eccentric contraction in which a muscle is contracted with the length being extended, and a concentric contraction in which a muscle is contracted with the length being shortened.

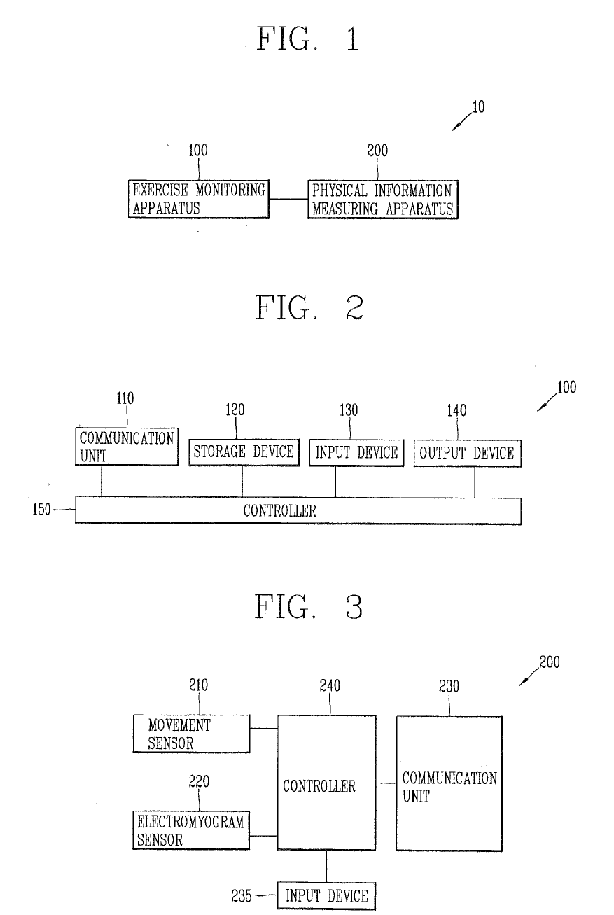

FIG. 1 is a block diagram of an exercise monitoring system in accordance with one exemplary embodiment. An exercise monitoring system in accordance with this embodiment may include an exercise monitoring apparatus 100 and a physical information measuring apparatus 200.

The physical information measuring apparatus 200 may measure a user's physical information using a sensor mounted or worn on the user's body or clothing. For instance, the physical information measuring apparatus 200 may generate a signal when a muscle movement is detected (muscle movement signal) via an inertial sensor affixed onto the body, and generate an electromyographic signal when electric activity is detected via an electromyographic sensor that may have one or more electrodes affixed to a muscular surface. The physical information measuring apparatus 200 may send the muscle movement signal and the detected electromyographic signal to the exercise monitoring apparatus 100. Here, the physical information apparatus 200 may periodically receive a transmission request from the exercise monitoring apparatus 100. In response, the physical information apparatus 200 may send the detected muscle movement related signal and electromyographic signal to the exercise monitoring apparatus 100.

The exercise monitoring apparatus 100 may analyze a user's exercise based upon the muscle movement related signal and the electromyographic signal received from the physical information measuring apparatus 200, and may store a result of the analysis or provide the same to the user. The exercise monitoring apparatus 100 may acquire muscle identification information and determine a type of exercise based on the acquired muscle identification information. Also, the exercise monitoring apparatus 100 may measure a level of muscle fatigue based on the muscle movement related signal, the type of exercise, and the electromyographic signal so as to store the measured muscle fatigue in a storage device or output the same via an output device, such as a display.

The physical information measuring apparatus 200 and the exercise monitoring apparatus 100 may be accessed via a wired/wireless network including an Internet network or an access interface. For access via the wired network, the physical information measuring apparatus 200 and the exercise monitoring apparatus 100 may be provided with an Ethernet terminal and the like. Also, various communication technologies may be used for access via the wireless network. Examples of the communication technologies may include Wireless LAN (WLAN) (Wi-Fi), Wireless Broadband (Wibro), Worldwide Interoperability for Microwave Access (Wimax), High Speed Downlink Packet Access (HSDPA), or another appropriate communication protocol. Also, examples of the access protocol may include BLUETOOTH, Radio Frequency IDentification (RFID), Infrared Data Association (IrDA), Ultra-WideBand (UWB), ZigBee, Digital Living Network Alliance (DLNA), and the like.

In one exemplary embodiment, the exercise monitoring apparatus 100 and the physical information measuring apparatus 200 may perform communications by adapting methods defined in ISO/IEEE 11073 PHD (Personal Health Device). In another exemplary embodiment, the exercise monitoring apparatus 100 may be implemented as a stationary terminal, such as an image display device, for example, a TV or the like, as well as a mobile terminal such as a cellular phone, smart phone, PDA, or the like. In addition, in one exemplary embodiment, a plurality of physical information measuring apparatuses 200 may be connected to one or more exercise monitoring apparatuses 100, while a plurality of exercise monitoring apparatuses 100 may be connected to one or more physical information measuring apparatuses 200.

FIG. 2 is a block diagram of an exercise monitoring apparatus as shown in FIG. 1. The exercise monitoring apparatus 100 according to one exemplary embodiment may include a communication unit or interface 110, a storage device 120, an input device 130, an output device 140 and a controller 150.

The communication unit 110 may be operable to allow communications between the exercise monitoring apparatus 100 and the physical information measuring apparatus 200. For example, the communication unit 110 may be operable to receive the muscle movement signal and the electromyographic signal from the physical information measuring apparatus 200 in a periodic manner. In addition, the communication unit 110 may be operable to send a request for the muscle movement signal and the electromyographic signal to the physical information measuring apparatus 200, and receive those signals from the physical information measuring apparatus 200. In one exemplary embodiment, the communication unit 110 may receive muscle identification information from the physical information measuring apparatus 200.

The storage device 120 may be operable to store a program to operate the controller 150 and may temporarily store input/output data. The storage device 120 may also store a level of muscle fatigue determined by the controller 150. The storage device 120 may store the muscle identification information acquired by the controller 150 or the determined type of exercise in association with the muscle fatigue.

The input device 130 may be operable to allow a user to generate input data for operation control of the exercise monitoring apparatus 100. The input device 130 may include a keypad, a dome switch, a touchpad (e.g., static pressure/capacitance), a jog wheel, a jog switch, or another appropriate type of an input interface. The input device 130 may also allow the user to select a function provided in the exercise monitoring apparatus 100, execute a command, input muscle identification information, or the like.

The output device 140 may be operable to generate outputs related to visual, audible and tactile senses, and include, a display, an audio output module, an alarm, a haptic module, or another appropriate type of output interface. The display or audio output module may be configured to output the level of muscle fatigue determined by the controller 150. Also, the display or audio output module may be configured to output the level of muscle identification information determined by the controller 150 or the determined type of exercise together with the level of muscle fatigue. The display or audio output module may also output a value for exercise intensity measured by the controller 150 or a level of exercise risks determined by the controller 150. The display or audio output module may output an alert message generated by the controller 150. The display or audio output module may output the alert message generated by the controller 150 via the alarm or haptic module.

The controller 150 may control an overall operation of the exercise monitoring apparatus 100. The controller 150 may control the communication interface 110 to send a transmission request for the muscle movement related signal and the electromyographic signal to the physical information measuring apparatus 200. Also, the controller 150 may acquire muscle identification information and check a type of exercise based upon the acquired muscle identification information. The controller 150 may also measure a level of muscle fatigue based upon the checked type of exercise and the muscle movement related signal and the electromyographic signal received via the communication unit 110.

Also, the controller 150 may store the muscle fatigue, the muscle identification information, the type of exercise and the like in the storage device 120. The controller 150 may receive the muscle identification signal input via the input device 130. The controller 150 may output the muscle fatigue, the exercise intensity, the type of exercise, the exercise risks, the alert message or the like via the output device 140.

FIG. 3 is a block diagram of a physical information measuring apparatus as shown in FIG. 1. The physical information measuring apparatus 200 in accordance with one exemplary embodiment may include a movement sensing unit 210, an electromyogram detecting unit 220, a communication unit 230 and a controller 240. Also, the physical information measuring apparatus 200 may further include an input device 235.

The movement sensing unit 210 may measure a type of muscle movement and/or duration of a movement generated in muscles by using an inertial sensor affixed or worn on a human body or clothes. In one exemplary embodiment, the inertial sensor may include an acceleration sensor or a gyro sensor.

The acceleration sensor is a device that converts an acceleration change in one direction into an electric signal. In general, the acceleration sensor may be configured to convert acceleration changes in three axes with respect to the movement of the physical information measuring apparatus 200 into electric signals, so as to allow measuring of the acceleration in each axis.

The gyro sensor is a sensor that measures an angular velocity of the physical information measuring apparatus 200 performing a rotary motion, which senses (detects) a rotated angle from each reference direction. For example, the gyro sensor may detect each rotation angle based upon three directional axes, namely, yaw, pitch and roll.

The exercise monitoring apparatus 100 may detect, from acceleration data detected from the muscle movement related signal, whether any movement is generated in the muscles, whether the movement generated in the muscles is a rotational movement, whether the rotational movement is an internal rotation or an external rotation, and the like. Also, the exercise monitoring apparatus 100 may detect, from angular velocity data detected from the muscle movement related signal, an existence of a centripetal acceleration (motion) generated by a rotation of the muscles, a centripetal direction, and the like.

The electromyogram detecting unit 220 may detect an electric signal (electromyographic signal) generated according to the level of muscle contraction by use of electrodes affixed onto the muscular surfaces. The electromyographic signal may include a recording of motion-unit-action-potentials conducted along muscular fibers, which may be generated when muscles are contracted or relaxed. Also, when muscles are fatigued, an amplitude of the signal may increase, resulting in an extension of the period. The analysis for muscle fatigue may be done using a root-mean-square (RMS) amplitude based upon the amplitude of the electromyographic signal, a Median frequency according to a frequency spectrum analysis, an average frequency, and the like.

The communication unit 230 may allow communications between the physical information measuring apparatus 200 and the exercise monitoring apparatus 100. The communication unit 230 may periodically send the muscle movement signal and the electromyographic signal to the exercise monitoring apparatus 100. Alternatively, the communication unit 230 may send the muscle movement signal and the electromyographic signal in response to the request by the exercise monitoring apparatus 100. Also, the communication unit 230 may periodically send muscle identification information to the exercise monitoring apparatus 100 in response to the request by the exercise monitoring apparatus 100.

The controller 240 may control operations of the movement sensing unit 210 (movement sensor), the electromyogram detecting unit 220 (electromyogram sensor) and the communication unit 230. For example, the controller 150 may amplify signals detected by the movement sensing unit 210 and/or the electromyogram detecting unit 220, and may convert the amplified signals into signals having a format to be sent to the exercise monitoring apparatus 100 via the communication unit 110. The controller 240 may send the converted signals to the exercise monitoring apparatus 100 via the communication unit 230.

The input device 235 may allow a user to generate input data to control the physical information measuring apparatus 200. In one exemplary embodiment, the input device 235 may allow a user to input the muscle identification information.

In the meantime, the physical information measuring apparatus 200 may further include a storage device which may store a program to operate the controller 240 and may temporarily store input/output data, and an output device that may generate visual, audible, tactile, another appropriate type of outputs based on a content of the output.

The embodiments as broadly described herein may be implemented in a computer-readable medium using, for example, software, hardware, or some combination thereof. For a hardware implementation, the embodiments described herein may be implemented within one or more Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, microcontrollers, microprocessors, other electronic units designed to perform the functions described herein, or a selective combination thereof. In some cases, such embodiments as described herein may be implemented in the controller 150, 240.

For a software implementation, certain embodiments of procedures and functions may be implemented together as separate software modules, each of which may perform at least one of the disclosed functions or operations. The software codes may be implemented with a software application written in any suitable programming language. Also, the software codes may be stored in the storage device and executed by the controller 150, 240.

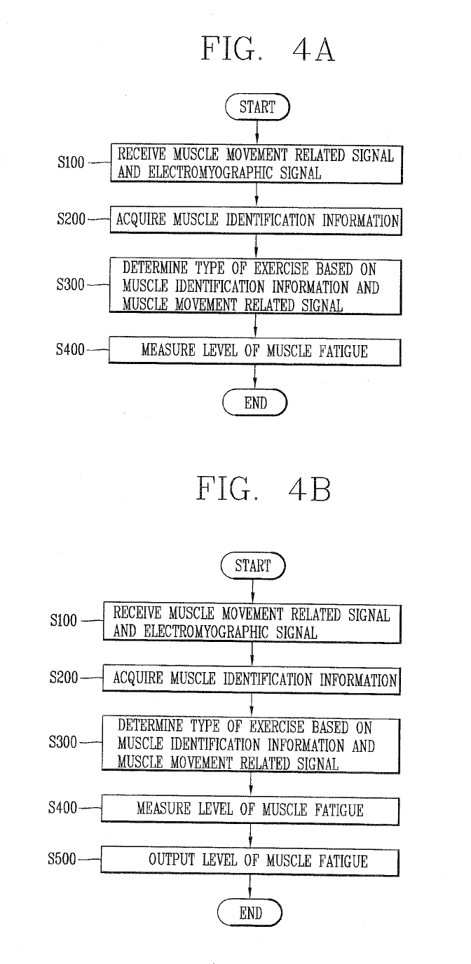

FIG. 4A is a flowchart of a method for monitoring exercise in accordance with a first exemplary embodiment. Referring to FIG. 4A, the exercise monitoring apparatus 100 may receive a signal related to muscle movements and an electromyographic signal from the physical information measuring apparatus 200, in step S100. The exercise monitoring apparatus 100 may also acquire muscle identification information, in step S200, and may determine a type of exercise based on the acquired muscle identification information and the muscle movement related signal, in step S300. Especially, the exercise monitoring apparatus 100 may receive the muscle identification information via the input device 130. In certain embodiments, the exercise monitoring apparatus 100 may receive the muscle identification information from the physical information measuring apparatus 200. For example, an input interface may be provided on the physical information measuring apparatus 200 such that the muscle identification information may be input at the sensor. Alternatively, the physical information measuring apparatus 200 may identify the muscle based on sensed muscle movement information obtained from the sensors. Once determined, the muscle identification information may be transmitted to the exercise monitoring apparatus 100.

In one exemplary embodiment, the controller 150 may determine the type of exercise, for example, a type of muscle contraction, based upon the muscle identification information and the muscle movement related signal. To this end, the controller 150 may extract acceleration data and angular velocity data from the muscle movement related signal.

For example, if a type of muscle is the biceps brachii and the extracted acceleration data indicates no changes in acceleration, the controller 150 may determine the type of exercise which the user is performing to be an isometric contraction. In this case, no change may be detected from the angular velocity data. Also, if the extracted acceleration data indicates that acceleration has changed, the controller 150 may determine the type of exercise the user is performing to be an isotonic contraction. In this case, the acceleration data may be divided into an adduction and an abduction.

If the acceleration is in a direction of the adduction and the angular velocity is a positive centripetal acceleration (motion), the controller 150 may determine the type of exercise to be a concentric contraction. Furthermore, if the acceleration is in a direction of the abduction and the angular velocity is a negative centripetal acceleration, the controller 150 may determine the type of exercise to be an eccentric contraction. In addition, when the type of muscle is a femoral muscle, if the extracted acceleration component is rotational and the angular velocity component contains a positive centripetal acceleration and a negative centripetal acceleration in a periodical manner, the controller 150 may determine the type of exercise to be an isokinetic contraction. The exercise monitoring apparatus 100 may then measure a level muscle fatigue based upon the type of exercise, the muscle movement related signal and the electromyographic signal, in step S400.

In accordance with the one exemplary embodiment, the controller 150 may measure the level of muscle fatigue by converting the electromyographic signal received from the physical information measuring apparatus 200 via the communication unit 110 into frequencies. Alternatively, the controller 150 may measure the level of muscle fatigue based upon the difference between an initial Median frequency and a final Median frequency, computed from the electromyographic signal received from the physical information measuring apparatus 200 via the communication unit 110. In one exemplary embodiment, the initial Median frequency and the final Median frequency may be Median frequencies computed at a start time and an end time within each muscle fatigue measuring section. The measuring section may be divided into a plurality of sections, and the initial Median frequency and the final Median frequency may be Median frequencies, computed at a start time and an end time of each divided section.

Meanwhile, when measuring the level of muscle fatigue by using only the electromyographic signal, for example, when no muscle movement is detected, a Median frequency estimated from the electromyographic signal generated may be low. Accordingly, in spite of the level of muscle fatigue not actually being high, the level of muscle fatigue may be measured as being in a high state. This problem may be caused when the Median frequency is generated as being low, for example, when the Median frequency is estimated from an electromyographic signal resulting from an instantaneous or temporary tension, twisting, or trembling of muscles. Consequently, the muscle fatigue is erroneously measured as being high.

In this embodiment, when the muscle movement related signal received from the physical information measuring apparatus 200 is used in the determination of a level of muscle fatigue, the exercise monitoring apparatus 100 may measure the muscle fatigue based on whether any movement has been detected from muscles (e.g., the exercise monitoring apparatus 100 may determine that the muscle movement is present when the detected muscle movement is higher than a threshold value for a predetermined time) and/or a duration of the muscle movement. Therefore, the exercise monitoring apparatus 100 may obviate or mitigate erroneous measurements of muscle fatigue levels caused by temporary tension, twisting, or trembling of the muscles, so as to accurately measure the actual muscle fatigue.

In one exemplary embodiment, the controller 150 may analyze a muscle movement related signal received from the physical information measuring apparatus 200 via the communication unit 110, and determine whether any muscle movement has been generated based upon the analyzed signal. For instance, during an isometric contraction exercise, the controller 150 may detect a minute trembling of muscles from the analyzed signal with respect to the muscle movement, and then measure a level of muscle fatigue from the electromyographic signal only when an excessive muscle contraction time (duration) which caused the muscle fatigue is higher than a threshold value, or measure a muscle fatigue by using the muscle contraction time (duration) as a weight value. As another example, during an isotonic contraction or isokinetic contraction exercise, the controller 150 may determine whether any muscle movement has been detected based upon the analyzed signal with respect to the muscle movement, and/or determine whether any muscle movement which may increase the muscle fatigue has been detected based upon the duration of the muscle movement. Alternatively, the controller 150 may measure a muscle fatigue based upon the electromyographic signal by determining a level (e.g., velocity and time) of the muscle movement and converting the determined movement level into a weight value.

FIG. 4B is a flowchart of a method for monitoring exercise in accordance with a second exemplary embodiment. Description of steps S100 to S400 is the same as the description of the steps S100 to S400 of FIG. 4A, as previously discussed and is omitted hereinbelow. In step S500, the controller 150 may output the measured muscle fatigue via the output device 140. In certain embodiments, the controller 150 may output the measured muscle fatigue via the display device or the audio output module. Moreover, in certain embodiments, the controller 150 may output not only the measured muscle fatigue but also muscle identification information and a type of exercise being performed.

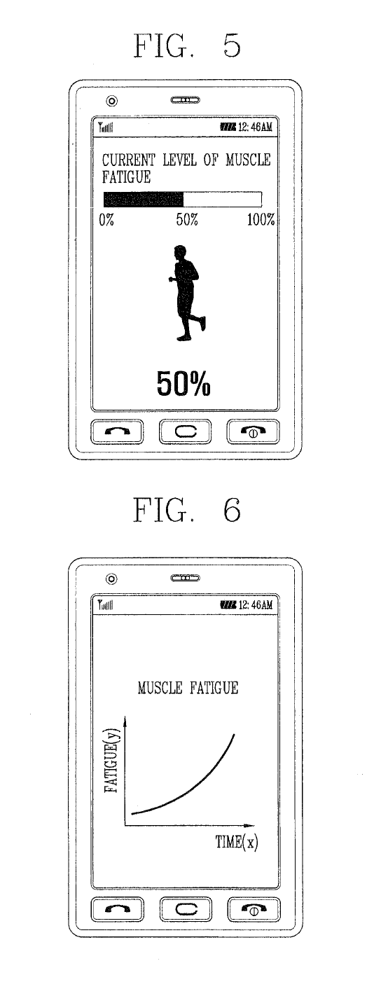

FIGS. 5 and 6 illustrate screens that display a level of muscle fatigue in accordance with the second exemplary embodiment. Referring to FIG. 5, the controller 150 may display a measured level of muscle fatigue (e.g., 50%) on a screen in a percentile form. In certain embodiments, the muscle fatigue may be a converted value of a fatigue degree of muscles, which may be measured by a difference between an initial Median frequency and a final Median frequency and a ratio of the initial Median frequency. Here, the muscle fatigue may be expressed as a percentage value in a text format. A higher muscle fatigue may indicate that the muscle is more fatigued. Also, the controller 150 may display the measured muscle fatigue (e.g., 50%) on a screen in an image form. In certain embodiments, the muscle fatigue in a range of 0% to 100% may be displayed in an image form, or a current muscle fatigue (e.g., 50%) may distinguishably be displayed within an image.

Referring to FIG. 6, the controller 150 may display the measured muscle fatigue in a graph form. In certain embodiments, the measured muscle fatigue may be expressed in a two-dimensional graph by setting X-axis to time and Y-axis to the muscle fatigue. In another exemplary embodiment, the controller 150 may display the measured muscle fatigue in a circular graph rather than a linear graph.

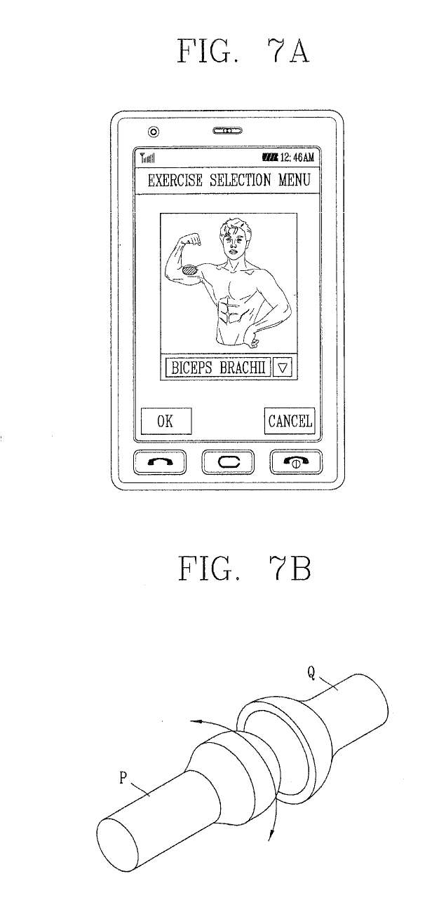

FIG. 7A illustrates a muscle input screen in accordance with one exemplary embodiment. As shown in FIG. 7A, the controller 150 may provide a user with a selection menu for a muscle whose fatigue is to be measured, and receive a user's selection with respect to the muscle for which fatigue is to be measured so as to acquire identification information related to the selected muscle. In another exemplary embodiment, the physical information measuring apparatus 200 may be provided with the input device 235 to receive an input to select a muscle for which fatigue is to be measured. In yet another embodiment, the physical information measuring apparatus 200 may determine the muscle identification information based on sensed motion of the muscles, as described further hereinbelow. Then, the communication unit 230 may send information related to the selected muscle to the exercise monitoring apparatus 100. Thus, the exercise monitoring apparatus 100 may acquire the information related to the selected muscle.

FIG. 7B illustrates a process of acquiring muscle identification information in accordance with one exemplary embodiment. Identification information related to a muscle for which fatigue is to be measured, as shown in FIG. 7A, may be acquired by a user input, and alternatively may be acquired based upon a signal output from a sensor namely, a signal related to the movement of a muscle onto which the sensor is affixed or worn. Muscles are moved by relaxing and contracting the muscles. Thus, muscle related identification information, which may be acquired from the muscle movement related signal, may be indirectly determined according to a shape or motion of a joint corresponding to the muscles attached thereto.

That is, the controller 150 may obtain identification information related to a joint attached with muscles, to which a sensor is substantially affixed or worn, based upon the muscle movement related signal received from the physical information measuring apparatus 200. The muscle identification information may also be obtained based upon the obtained joint related identification information.

Joints may be classified into immovable joints (immovable articulation, synarthrosis) and movable joints (movable articulation, synovial joints). The movable joints as mobility joints may be divided according to the figure of an articular facet into plane joints, hinge joints, pivot (trochoid) joints, ellipsoidal joints, saddle joints and ball-and-socket joints. The plane joints are found, for example, in the wrists and opposed surfaces of the bones are almost flat with limited movements. The hinge joint is a bone joint, for example, a joint between finger bones, in which the articular surfaces are molded to each other in such a manner as to permit only two motions, namely, folding and unfolding motions. The pivot joint is a joint, for example, proximal radiulnar joint, in which a bone with a circular articular head can uniaxially rotate along a bone with a glenoid cavity. The ellipsoidal joint is a joint, for example, radiocarpal joint, in which two articular surfaces ovally rotate and are movable in two directions, namely, in a long axis and a short axis. The saddle joint, such as carpometacarpal articulation of the thumb, has an articular surface in a shape of a saddle, and is a biaxial joint which is movable perpendicularly. The ball-and-socket joint has an articular head in a shape of a ball, and glenoid cavity thereof is in a shape of a mortar, allowing greater freedom of movement and multiaxial movement.

Referring to FIG. 7B, the ball-and-socket joints may be formed such that the articular head P is in the shape of a ball and the glenoid cavity is in the shape of a mortar, thereby allowing greater freedom of movement and mutliaxial movement. The controller 150 may analyze the muscle movement related signal for movement of the muscle and corresponding joint. If the movement is done freely and multiaxially, the controller 150 may determine the joint associated with the sensor-affixed joint as the ball-and-socket joint (e.g., glenohumeral joint or tibiofemoral joint). Consequently, the controller 150 may acquire identification information related to the muscles associated with the glenohumeral joint or tibiofemoral joint.

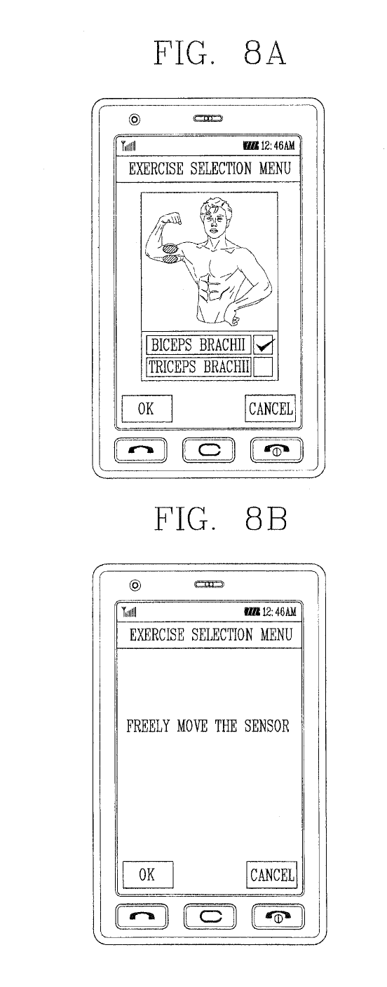

FIG. 8A illustrates a muscle selection screen in accordance with one exemplary embodiment. Referring to FIG. 8A, if a plurality of joints are identified by the controller 150, or a plurality of muscles are attached onto the identified joints, the controller 150 may output information related to the muscles attached to the identified joints through the display device. Accordingly, a plurality of muscles to which the sensor may be affixed may be filtered, such that a user may input the muscle related identification information more conveniently. For example, if the joint identified by the controller 150 is the glenohumeral joint, information related to muscles attached onto the glenohumeral joint may be output on the display, and menus, from which the user may actually select the sensor-affixed muscle from the displayed muscles, may also be output on the display. Those menus may be displayed at positions which correspond to the filtered muscles on an image of a human body (e.g., actual image of the user or a virtual image), so as to be provided to the user in a more intuitive manner. The controller 150 may acquire the muscle identification information based upon the muscle selected by the user.

FIG. 8B illustrates a screen that displays a user prompt in accordance with one exemplary embodiment. Referring to FIG. 8B, the controller 150 may generate a user prompt or guide information related to a reference motion for filtering muscles whose fatigue is to be measured, and output the generated user prompts via the output device 140. Here, the reference motion may indicate a movement by which muscles are identifiable, and may be a combination of several motions. For example, the reference motion may include a three-dimensional motion at a sensor-affixed portion. The controller 150 may acquire identification information related to muscles for which fatigue is to be measured based upon the muscle movement related signal received from the physical information measuring apparatus 200 of the user who performs the reference motion. The reference motion, as shown in FIG. 8B, may include motions within a maximum activity range of muscles associated with the sensor-affixed portion.

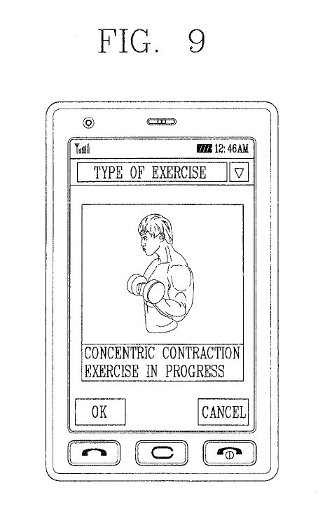

FIG. 9 illustrates a screen that displays a type of exercise in accordance with the second exemplary embodiment. Referring to FIG. 9, the controller 150 may determine a type of exercise based upon the acquired muscle identification information and the muscle movement related signal received from the physical information measuring apparatus 200, and output the determined type of exercise on the display. For example, if the input portion in motion is the biceps brachii, the acceleration component and the angular velocity component extracted from the muscle movement related signal may indicate adduction and positive centripetal acceleration, respectively. The controller 150 may determine the type of exercise as a concentric contraction, and display on a screen that the concentric contraction exercise is in progress or being performed.

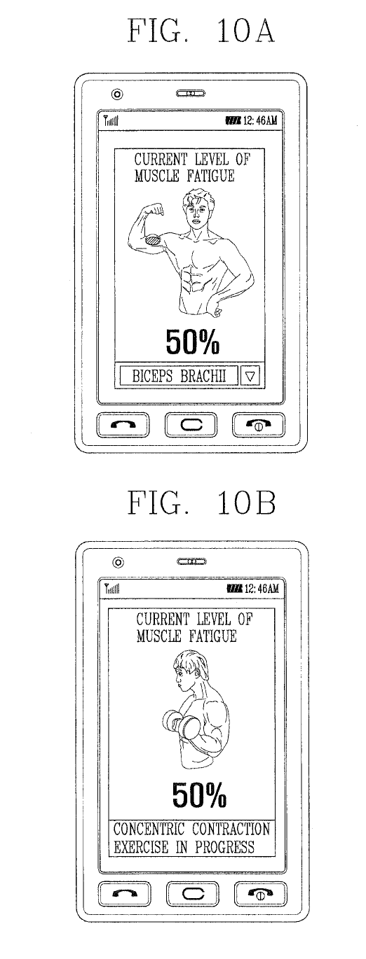

FIGS. 10A and 10B illustrate screens that display a level of muscle fatigue in accordance with the second exemplary embodiment. Referring to FIG. 10A, the controller 150 may display the muscle identification information together with a measured muscle fatigue (e.g., 50%). Hence, the user may identify muscles that the user is using and monitor the level of fatigue of the identified muscles based upon the muscle identification information and the level of muscle fatigue provided by the exercise monitoring apparatus 100.

Referring to FIG. 10B, the controller 150 may display the type of exercise together with the measured muscle fatigue (e.g., 50%). Hence, the user may identify the type of exercise that the user is performing and monitor the level of muscle fatigue with respect to the identified type of exercise based upon the type of exercise and the level of muscle fatigue provided by the exercise monitoring apparatus 100.

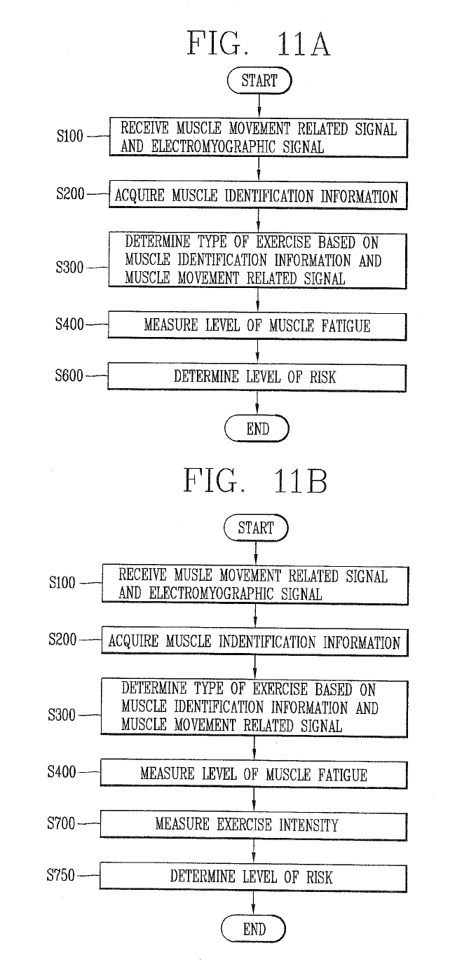

FIG. 11A is a flowchart of a method for monitoring exercise in accordance with a third exemplary embodiment. Description of steps S100 to S400 is the same as the description of the steps S100 to S400 of FIG. 4A, as previously described and is omitted hereinbelow. The exercise monitoring apparatus 100 may compare the measured muscle fatigue with a threshold value corresponding to the determined type of exercise, to determine the risk of the exercise, in step S600. Also, the exercise monitoring apparatus 100 may output the risk via the output device 140.

Here, a threshold value for determination of the risk may be a muscle fatigue measured, with respect to an ordinary user, according to a type of exercise (or muscle identification information and a type of exercise) in a stable state. The threshold value may be previously stored in the storage device 120. Here, for more accurate determination of the risk, a muscle fatigue, which is previously measured according to a type of exercise (or muscle identification information and a type of exercise) in a stable state with respect to a user who is a target for the risk determination, may be used as a threshold value. Since the number of muscle cells, volume and position of a muscle, which is a target for measuring an exercise risk, depend on an individual person, the threshold value as the reference for determining the risk may be different individually.

In this case, the controller 150 may provide a menu to measure the muscle fatigue according to the type of exercise (or muscle identification information and a type of exercise) in a stable state with respect to the user, who is the target for the risk determination, store the muscle fatigue measured via the menu selection in the storage device 120, and use the stored muscle fatigue as a threshold value. Also, upon change in the number of muscle cells, the volume and the position of the user's muscle, the user's muscle fatigue may be re-measured according to a type of exercise (or muscle identification information and a type of exercise) in a stable state, and the re-measured muscle fatigue may be updated in the storage device 120 to be used as a threshold value.

FIG. 11B is a flowchart of a method for monitoring exercise in accordance with a fourth exemplary embodiment. Description of steps S100 to S400 is the same as the description of the steps S100 to S400 of FIG. 4A, as previously described and is omitted hereinbelow. The exercise monitoring apparatus 100 may measure an exercise intensity level through an Integrated-Electromyogram (I-EMG) analysis for the electromyographic signal, in step S700. Also, the exercise monitoring apparatus 100 may determine a first risk of the exercise by comparison of the measured muscle fatigue with a threshold value, and then determine a second risk of the exercise based upon the muscle movement related signal and the measured exercise intensity, in step S750. The exercise monitoring apparatus 100 may determine the risk of the exercise based upon the first risk and the second risk. Also, the exercise monitoring apparatus 100 may store the measured exercise intensity in the storage device 120 and/or output the same via the output device 140. Alternatively, the exercise monitoring apparatus 100 may output the risk via the output device 140.

In accordance with one exemplary embodiment, the controller 150 may measure exercise intensity through the I-EMG analysis for the electromyographic signal received from the physical information measuring apparatus 200. For example, the controller 150 may measure the exercise intensity by integrating the electromyographic signal with a muscle contraction time (duration). That is, I-EMG may be the sum of signals exhibited during the muscle contraction. That is, an activity level during the muscle contraction may be taken as the exercise intensity. In certain embodiments, the controller 150 may apply an absolute or rectified value to the electromyographic signal received from the physical information measuring apparatus 200 to rectify only bidirectional components and then may level the rectified values. Also, the controller 150 may integrate the leveled value with an axis of time in a leveled graph so as to measure exercise intensity.

In the meantime, the controller 150, as shown in FIG. 9A, may determine the first risk of the exercise based upon the muscle movement related signal and the electromyographic signal. Also, the controller 150, as aforementioned, may measure the exercise intensity level through the I-EMG analysis for the electromyographic signal and may compare the exercise intensity with the muscle movement related signal to determine the second risk. The controller 150 may determine the second risk according to the degree of the exercise intensity matching the degree of the muscle movement. In certain embodiments, if the exercise intensity pattern according to the elapse of time does not match the muscle movement pattern according to the elapse of time, the controller 150 may determine the ongoing exercise to be dangerous. Also, when the unmatched level is greater, the controller 150 may determine the exercise to be much more dangerous. Here, the controller 150 may determine the risk of exercise by comparing the variation of the exercise intensity with the variation of the movement level (which may indicate a physical value, such as velocity, acceleration, etc.) for a predetermined time.

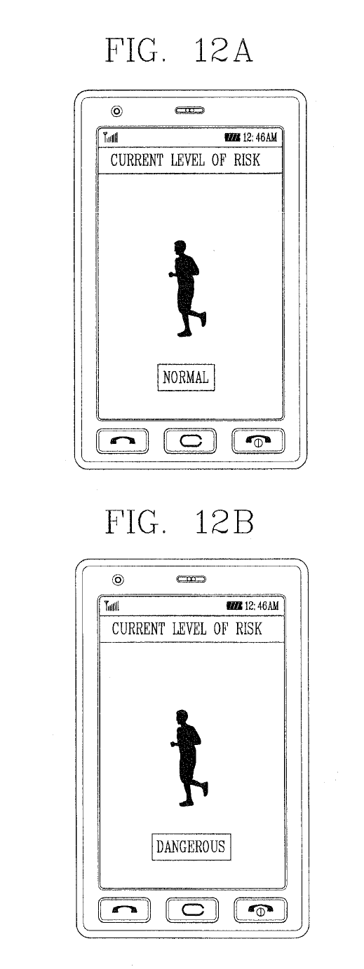

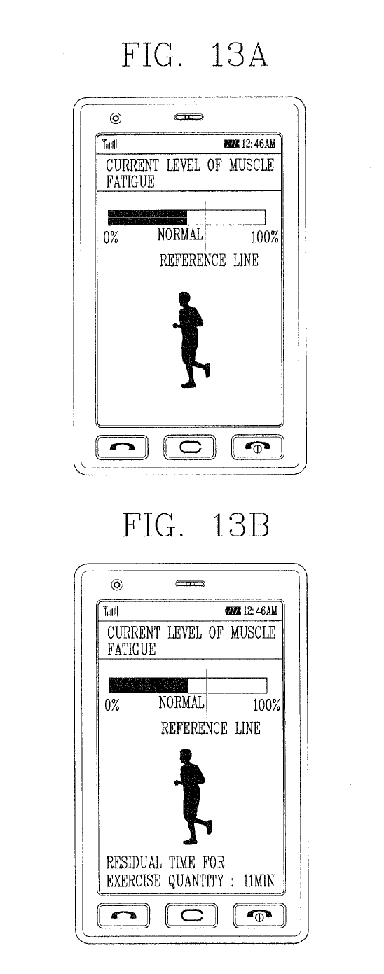

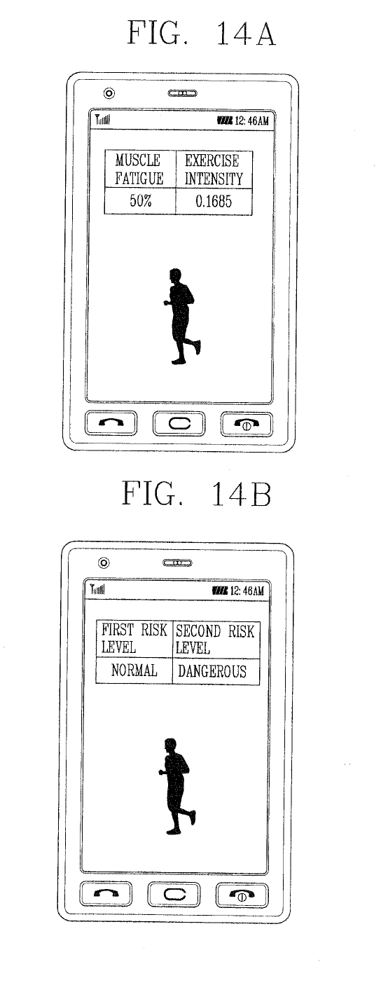

FIGS. 12 and 13 illustrate outputs of the exercise monitoring apparatus according to the method for monitoring exercise in accordance with the third exemplary embodiment. Referring to FIGS. 12A and 12B, the controller 150 may display the determined exercise risk (e.g., normal or dangerous) on a screen in a text format. In one exemplary embodiment, if a muscle fatigue measured during exercise does not exceed a threshold value by predetermined ratio, the controller 150 may determine the current exercise to be harmless, and display the determined risk (e.g., normal) on the screen in the text format. Also, if the muscle fatigue measured during the exercise exceeds the threshold value by the predetermined ratio, the controller 150 may determine the current exercise to be dangerous and display the determined risk (e.g., dangerous) on the screen in the text format.

Referring to FIGS. 13A and 13B, the controller 150 may display the determined exercise risk on the screen in a bar-like shape. In certain embodiments, a level of muscle fatigue in the range of 0% to 100% may be displayed in the bar-like shape, and the current level of muscle fatigue and the exercise risk (e.g., normal) may be distinguishably displayed in the bar. Here, a reference line, which may serve as a reference for the exercise risk based on the threshold value, may also be displayed. The user may monitor the risk associated with the current exercise and estimate an amount of time for which the ongoing exercise may be continued. In the meantime, referring to FIG. 13B, a residual time up to the reference line when the ongoing exercise pattern is continued may further displayed on the screen. In certain embodiments, the controller 150 may determine the residual time up to the reference line based upon the pattern of the muscle fatigue that has been measured since the start of the exercise.

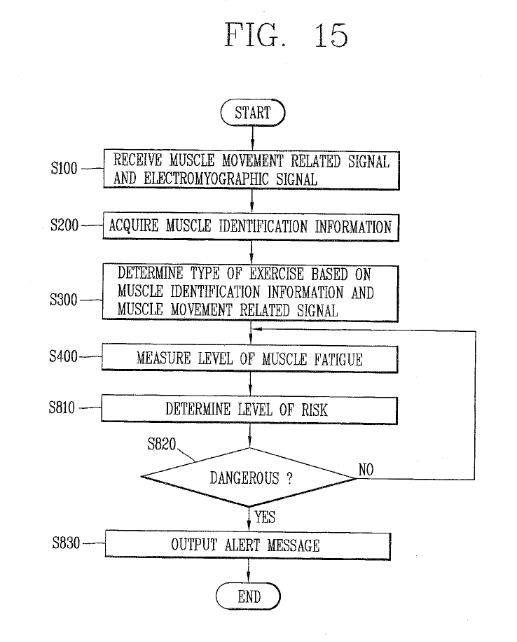

FIG. 14 illustrates an output of the exercise monitoring apparatus according to the method for monitoring exercise in accordance with the fourth exemplary embodiment. Referring to FIG. 14A, the controller 150 may display the measured level of muscle fatigue (e.g., 50%) on the screen as a percentage value in a text format. Also, the controller 150 may display the measured exercise intensity (e.g., 0.1685) on the screen with numerals in the text format.

Referring to FIG. 14B, the controller 150 may display a first risk level (e.g., normal or dangerous) of the exercise, determined based upon the muscle fatigue and the muscle movement related signal, on the screen in the text format. Also, the controller 150 may display a second risk level (e.g., normal or dangerous) of the exercise, determined based upon the exercise intensity and the muscle movement related signal, on the screen in the text format.

The controller 150 may also output the exercise risk based upon the determined first and second risk levels via the output device 140. In one exemplary embodiment, if one of the first and second risk levels indicate ‘dangerous,’ the controller 150 may determine the risk of the exercise to be dangerous. Also, if both the first and second risks indicate ‘normal,’ the controller 150 may determine the exercise risk to be normal.

FIG. 15 is a flowchart of a method for monitoring exercise in accordance with a fifth exemplary embodiment. Description of steps S100 to S400 is the same as the description of the steps S100 to S400 of FIG. 4A, as previously described and is omitted hereinbelow. The exercise monitoring apparatus 100 may generate an alert message based upon the risk and output the generated alert message via the output device 140. In one exemplary embodiment, the controller 150 may determine the exercise risk, in step S810, determine whether the determined exercise risk indicates danger, in step S820, and generate an alert message when the determined exercise risk is determined as being dangerous so as to output the same, in step S830.

FIG. 16 illustrates an output of the exercise monitoring apparatus according to the method for monitoring exercise in accordance with the fifth exemplary embodiment. Referring to FIG. 16, when the ongoing exercise is determined to be dangerous, the controller 150 may generate an alert message indicating that it is dangerous to continue the exercise, and output the generated alert message on the display device. In certain embodiments, the alert message may be audibly output via the audio output module. Alternatively, the alert message may be tactually output via the haptic module.

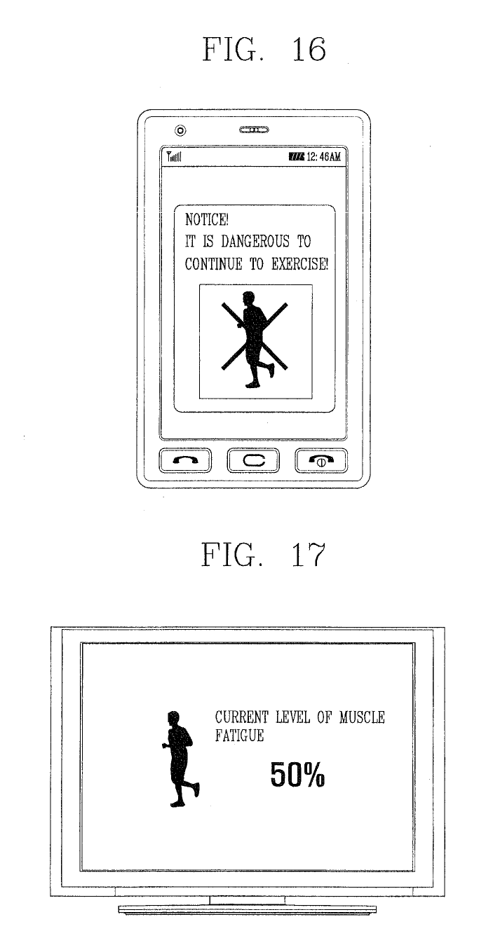

FIG. 17 illustrates an output of the exercise monitoring apparatus in accordance with a sixth exemplary embodiment. Referring to FIG. 17, if the exercise monitoring apparatus 100 is an image display device, the controller 150 may output the measured muscle fatigue level on the screen. As such, the exercise monitoring process in accordance with those exemplary embodiments may be implemented in stationary terminals including the image display device, such as a TV or the like.

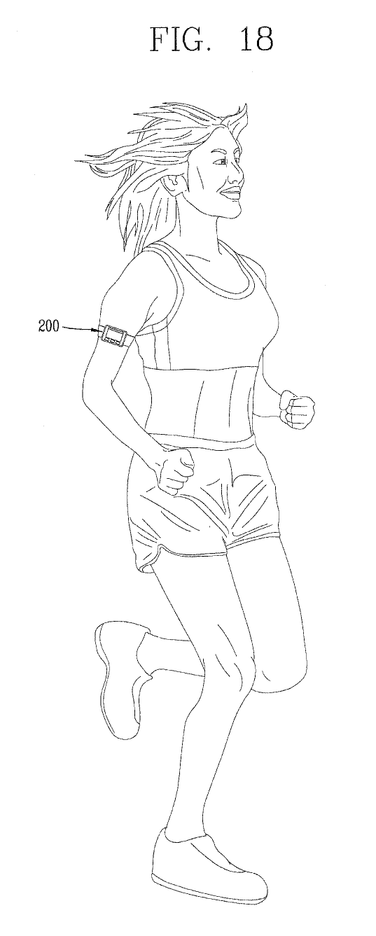

FIG. 18 is a conceptual view showing example to mount the physical information measuring apparatus according to one embodiment of the present invention. The physical information measuring apparatus 200 may have a shape suitable for being mounted to a human's body or clothes.

Referring to FIG. 18, the physical information measuring apparatus 200 may be a type attachable to an arm band (or wrist band, etc.) mounted onto a user's arm (or wrist, etc.). The physical information measuring apparatus 200 may detect the muscle movement signal via an inertial sensor affixed onto the body when a muscle movement is detected. Also, when an electric activity is detected, the physical information measuring apparatus 200 may detect the electromyographic signal via an electromyographic sensor that may have one or more electrodes affixed to a muscular surface.

Based on these signals, the exercise monitoring apparatus 100 may determine a muscle fatigue level.

An exercise monitoring apparatus, as embodied and broadly described herein, may include a communication unit adapted to receive a muscle movement signal and an electromyographic signal from a physical information measuring apparatus; and a controller adapted to acquire muscle identification information, determine a type of exercise based upon the acquired identification information and the muscle movement signal, and determine a muscle fatigue level based on the type of exercise, the muscle movement signal, and the electromyographic signal.

The apparatus may further include a storage device adapted to store the determined muscle fatigue level, wherein the storage device stores the muscle identification information or the determined type of exercise associated with the determined muscle fatigue level, and may further include an output device adapted to output the determined muscle fatigue level, and wherein the output device outputs the muscle identification information or the determined type of exercise together with the determined muscle fatigue level.

In this exercise monitoring apparatus, the controller may determine a level of risk based on a threshold value of muscle fatigue level corresponding to the type of exercise and the determined muscle fatigue level. Moreover, this apparatus may further include an output device adapted to output the determined level of risk, and an output device adapted to output an alert message based on the determined level of risk, wherein the controller measures an exercise intensity level through an Integrated-EMG (I-EMG) analysis for the electromyographic signal, and wherein the controller determines a level of risk associated with the exercise based on the muscle movement signal and the measured exercise intensity level. This exercise monitoring apparatus may further include a storage device adapted to store the measured exercise intensity level, and an output device adapted to output the measured exercise intensity level.

This exercise monitoring apparatus may further include an input device adapted to receive an input of the muscle identification information, wherein the communication unit receives the muscle identification information from the physical information measuring apparatus. Moreover, in this embodiment, the controller may identify a joint associated with the muscle movement signal and one or more muscles which are attached to the identified joint, and wherein the muscle identification information is determined based on the identified joint and the one or more muscles attached to the joint. Furthermore, in this apparatus, if a plurality of joints are identified or if a plurality of muscles are attached to the identified joint, the controller is configured to determine the muscle identification information based on muscles attached to the plurality of identified joints or the plurality of muscles attached to the identified joint, wherein the output device is configured to display a user prompt to instruct a user to move a sensor related to the muscle to acquire the muscle identification information. Moreover, the muscle movement signal may be detected by an acceleration sensor or a gyro sensor.

A method for monitoring exercise, as embodied and broadly described herein, may include receiving a muscle movement signal and an electromyographic signal from a physical information measuring apparatus; acquiring a muscle identification information related to the muscle movement signal; determining a type of exercise based on the acquired muscle identification information and the muscle movement signal; and determining a level of muscle fatigue based on the type of exercise, the muscle movement signal, and the electromyographic signal.

An exercise monitoring system, as embodied and broadly described herein, may include a physical information measuring apparatus and an exercise monitoring apparatus, wherein the physical information measuring apparatus includes at least one sensor configured to generate a muscle movement related signal and an electromyographic signal, and a first communication interface adapted to transmit the muscle movement related signal and the electromyographic signal to the exercise monitoring apparatus. In this embodiment, the exercise monitoring apparatus may include a second communication interface configured to receive the muscle movement related signal and the electromyographic signal from the physical information measuring apparatus, and a controller configured to identify a muscle corresponding to the muscle movement related signal, determine a type of exercise based on the identified muscle and the muscle movement related signal, and measure a level of muscle fatigue based on the type of exercise, the muscle movement related signal, and the electromyographic signal.

An exercise monitor, as embodied and broadly described herein, may include a first sensor to detect a movement of a muscle; a second sensor to detect an electric activity in the muscle; a controller to receive the detected movement and electric activity from the first and second sensors, wherein the controller is configured to determine at least one of an amount of muscle fatigue, a type of muscle being monitored, a type of exercise being performed, exercise intensity, or health risks of a user based on the detected movement and electric activity; and a display that displays the amount of muscle fatigue and health risks. In this embodiment, the first sensor includes an accelerometer that detects an acceleration and a gyro sensor that detects an angular velocity, wherein the second sensor includes one or more probes that detect electromyographic signals.

Moreover, an exercise monitoring apparatus is further embodied and broadly disclosed herein which may include a communication unit adapted to receive a signal related to a muscle movement and an electromyographic signal from a physical information measuring apparatus, and a controller adapted to acquire identification information related to the muscle, determine a type of exercise based upon the acquired identification information and the muscle movement related signal, and measure a muscle fatigue based upon the type of exercise, the muscle movement related signal and the electromyographic signal.

In certain embodiments, the apparatus may further include a storage unit adapted to store the measured muscle fatigue. In certain embodiments, the storage unit may store the muscle identification information or the determined type of exercise in associated with the muscle fatigue. The apparatus may further include an output unit adapted to output the measured muscle fatigue. In an embodiment, the output unit may output the muscle identification information of the determined type of exercise together with the muscle fatigue. Moreover, in certain embodiments, the controller may determine a risk of the exercise by comparing a threshold value corresponding to the type of exercise with the measured muscle fatigue.

In certain embodiments, the controller may measure exercise intensity through an Integrated-EMG (I-EMG) analysis for the electromyographic signal. In an exemplary embodiment, the controller may determine a risk of the exercise based upon the muscle movement related signal and the measured exercise intensity. Moreover, the apparatus may further include a storage unit adapted to store the measured exercise intensity. In another exemplary embodiment, the apparatus may further include an output unit adapted to output the measured exercise intensity.

In certain embodiments, the apparatus may further include an output unit adapted to output the determined risk. In an exemplary embodiment, the apparatus may further include an output unit adapted to output an alert message based upon the determined risk. The apparatus may further include an input unit adapted to allow inputting of the muscle identification information.

In certain embodiments, the communication unit may receive the muscle identification information from the physical information measuring apparatus. In certain embodiments, the controller may identify a joint associated to the movement based upon the muscle movement related signal, determine a muscle attached onto the joint, and acquire identification information related to the muscle. In one exemplary embodiment, if the identified joint or the muscle attached onto the identified joint is in plurality, the controller may acquire identification information related to a muscle selected from the muscles attached onto the identified joint. In one exemplary embodiment, the output unit may output guide information related to the muscle movement for identification of the muscle. In certain embodiments, the muscle movement related signal may be detected by an acceleration sensor or a gyro sensor.

Moreover, to achieve the aspect of the detailed description as broadly described herein, there is provided an exercise monitoring method that may include receiving a signal related to a muscle movement and an electromyographic signal from a physical information measuring apparatus, acquiring identification information related to the muscle, determining a type of exercise based upon the acquired identification information and the muscle movement related signal, and measuring a muscle fatigue based upon the type of exercise, the muscle movement related signal and the electromyographic signal.

Moreover, to achieve the aspect of the detailed description as broadly described herein, there is provided an exercise monitoring system that may include a physical information measuring apparatus and an exercise monitoring apparatus, wherein the physical information measuring apparatus may include at least one sensor adapted to detect a muscle movement related signal and an electromyographic signal, and a communication unit adapted to send the muscle movement related signal and the electromyographic signal to the exercise monitoring apparatus, wherein the exercise monitoring apparatus may includes a communication unit adapted to receive the muscle movement related signal and the electromyographic signal from the physical information measuring apparatus, and a controller adapted to acquire identification information related to the muscle, determine a type of exercise based upon the acquired identification information and the muscle movement related signal, and measure a muscle fatigue based upon the type of exercise, the muscle movement related signal and the electromyographic signal.

In accordance with an exemplary embodiment, when a user keeps doing a muscular motion for a long time or a sudden load occurs in muscular cells to cause an immoderate muscle contraction, the fatigue affecting the muscles can be monitored, thereby obviating or eliminating risks, such as muscle cramp or muscle rupture, due to the immoderate muscular motion. Especially, use of inertial sensor allows an accurate measurement of a muscle fatigue in response to a user's movement, and the measured muscle fatigue can be utilized in various aspects, such as being associated with exercise intensity, determining a type of exercise according to a portion in motion and the like.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An exercise monitoring apparatus comprising:
a communication unit adapted to receive a muscle movement signal and an electromyographic signal from a physical information measuring apparatus; and
a controller adapted to acquire muscle identification information, determine a type of exercise based upon the acquired identification information and the muscle movement signal, and determine a muscle fatigue level based on the type of exercise, the muscle movement signal, and the electromyographic signal.

2. The exercise monitoring apparatus of claim 1, further comprising a storage device adapted to store the determined muscle fatigue level.

3. The exercise monitoring apparatus of claim 2, wherein the storage device stores the muscle identification information or the determined type of exercise associated with the determined muscle fatigue level.

4. The exercise monitoring apparatus of claim 1, further comprising an output device adapted to output the determined muscle fatigue level.

5. The exercise monitoring apparatus of claim 4, wherein the output device outputs the muscle identification information or the determined type of exercise together with the determined muscle fatigue level.

6. The exercise monitoring apparatus of claim 1, wherein the controller determines a level of risk based on a threshold value of muscle fatigue level corresponding to the type of exercise and the determined muscle fatigue level.

7. The exercise monitoring apparatus of claim 6, further comprising an output device adapted to output the determined level of risk.

8. The exercise monitoring apparatus of claim 6, further comprising an output device adapted to output an alert message based on the determined level of risk.

9. The exercise monitoring apparatus of claim 1, wherein the controller measures an exercise intensity level through an Integrated-EMG (I-EMG) analysis for the electromyographic signal.

10. The exercise monitoring apparatus of claim 9, wherein the controller determines a level of risk associated with the exercise based on the muscle movement signal and the measured exercise intensity level.

11. The exercise monitoring apparatus of claim 9, further comprising a storage device adapted to store the measured exercise intensity level.

12. The exercise monitoring apparatus of claim 9, further comprising an output device adapted to output the measured exercise intensity level.

13. The exercise monitoring apparatus of claim 1, further comprising an input device adapted to receive an input of the muscle identification information.

14. The exercise monitoring apparatus of claim 1, wherein the communication unit receives the muscle identification information from the physical information measuring apparatus.

15. The exercise monitoring apparatus of claim 1, wherein the controller identifies a joint associated with the muscle movement signal and one or more muscles which are attached to the identified joint, and wherein the muscle identification information is determined based on the identified joint and the one or more muscles attached to the joint.

16. The exercise monitoring apparatus of claim 15, wherein, if a plurality of joints are identified or if a plurality of muscles are attached to the identified joint, the controller is configured to determine the muscle identification information based on muscles attached to the plurality of identified joints or the plurality of muscles attached to the identified joint.

17. The exercise monitoring apparatus of claim 15, wherein the output device is configured to display a user prompt to instruct a user to move a sensor related to the muscle to acquire the muscle identification information.

18. The exercise monitoring apparatus of claim 1, wherein the muscle movement signal is detected by an acceleration sensor or a gyro sensor.

19. A method for monitoring exercise comprising:
receiving a muscle movement signal and an electromyographic signal from a physical information measuring apparatus;
acquiring a muscle identification information related to the muscle movement signal;
determining a type of exercise based on the acquired muscle identification information and the muscle movement signal; and
determining a level of muscle fatigue based on the type of exercise, the muscle movement signal, and the electromyographic signal.

20. An exercise monitoring system comprising:
a physical information measuring apparatus and an exercise monitoring apparatus,
wherein the physical information measuring apparatus includes
at least one sensor configured to generate a muscle movement related signal and an electromyographic signal, and
a first communication interface adapted to transmit the muscle movement related signal and the electromyographic signal to the exercise monitoring apparatus, and
wherein the exercise monitoring apparatus includes
a second communication interface configured to receive the muscle movement related signal and the electromyographic signal from the physical information measuring apparatus, and
a controller configured to identify a muscle corresponding to the muscle movement related signal, determine a type of exercise based on the identified muscle and the muscle movement related signal, and measure a level of muscle fatigue based on the type of exercise, the muscle movement related signal, and the electromyographic signal.

21. An exercise monitor comprising:
a first sensor to detect a movement of a muscle;
a second sensor to detect an electric activity in the muscle;
a controller to receive the detected movement and electric activity from the first and second sensors, wherein the controller is configured to determine at least one of an amount of muscle fatigue, a type of muscle being monitored, a type of exercise being performed, exercise intensity, or health risks of a user based on the detected movement and electric activity; and
a display that displays the amount of muscle fatigue and health risks.

22. The exercise monitor of claim 21, wherein the first sensor includes an accelerometer that detects an acceleration and a gyro sensor that detects an angular velocity.

23. The exercise monitor of claim 21, wherein the second sensor includes one or more probes that detect electromyographic signals.
